(19) United States
(12) Patent Application Publication
(10) Pub. No.: US 2007/0178137 A1
(43) Pub. Date: Aug. 2, 2007

(54) Local control of inflammation

(76) Inventors: Toby Freyman, Waltham, MA (US); Wendy Naimark, Cambridge, MA (US); Maria Palasis, Wellesley, MA (US)

Correspondence Address: COOLEY GODWARD KRONISH LLP;ATTN: PATENT GROUP, Suite 500, 1200 - 19th Street, NW, WASHINGTON, DC, 20036-2402, US

(21) Appl. No.: 11/344,156

(22) Filed: Feb. 1, 2006

(51) Int. Cl. A61K 35/12; A61K 38/20; A61K 38/13; A61K 31/573; A61K 31/4745; A61K 38/44; A61K 31/192

(52) U.S. Cl. 424/426; 514/171; 514/11; 424/93.7; 424/85.2; 424/94.4; 514/165; 514/569; 514/570; 514/291

(57) ABSTRACT

A medical device includes a carrier and an agent. The agent is formulated to control inflammation of biological tissue, such as heart tissue, and is releasably coupled to the carrier. The carrier is configured to be disposed in operative proximity to the biological tissue to be treated by the agent. In one embodiment, the carrier is configured to release the agent or otherwise deliver the agent to the biological tissue, thus controlling inflammation of the tissue. Also, a method to improve healing of biological tissue includes placing a medical device proximate to the heart of a patient, where the medical device has a carrier and an agent configured to control inflammation, the agent is releasably coupled to the carrier. In one embodiment, the method includes causing the agent to be released from the carrier.

### BACKGROUND

This invention relates generally to a medical device, and particularly to a medical device configured to be placed in or near the heart. This invention also relates to a method to improve healing of tissue.

Inflammation is a natural and necessary part of a body's healing process. However, this process has been increasingly linked with pathological and detrimental conditions, and even with disease. The natural inflammatory process that occurs after a myocardial infarction results in removal of the existing myocardial scaffold and ultimately leads to scar formation—a mechanically and functionally inferior tissue.

Systemic therapies that control inflammation of heart tissue or the biological tissue surrounding the heart have shown promise in treating heart disease. For example, better biological tissue may form if inflammation is controlled. However, such systemic therapies do not locally control inflammation of the heart tissue or the biological tissue surrounding the heart. Accordingly, there is a need for a device configured to locally control inflammation of heart tissue or the biological tissue surrounding the heart.

### SUMMARY

A medical device includes a carrier and an agent. The agent is formulated to control inflammation of biological tissue, such as heart tissue, and is releasably coupled to the carrier. The carrier is configured to be disposed in operative proximity to the biological tissue to be treated by the agent. In one embodiment, the carrier is configured to release the agent or otherwise deliver the agent to the biological tissue, thus controlling inflammation of the tissue.

A method to improve healing of biological tissue includes placing a medical device proximate to the heart of a patient, where the medical device has a carrier and an agent configured to control inflammation, the agent is releasably coupled to the carrier. In one embodiment, the method includes causing the agent to be released from the carrier.

### BRIEF DESCRIPTION OF THE DRAWINGS

FIG. 1 is a schematic illustration of a medical device according to an embodiment of the invention.

FIG. 2a is a perspective view of a medical device according to another embodiment of the invention.

FIG. 2b is a cross-sectional view of the medical device of FIG. 2a taken along line 2b-2b.

FIG. 2c is a cross-sectional view of a medical device according to another embodiment of the invention.

FIG. 3a is the top view of a medical device according to another embodiment of the invention.

FIG. 3b is the bottom view of the medical device of FIG. 3a.

FIG. 3c is a cross-sectional view of the medical device of FIG. 3b taken along line 3c-3c.

FIG. 4a is a perspective view of a medical device according to another embodiment of the invention.

FIG. 4b is a cross-sectional view of the medical device of FIG. 4a taken along line 4b-4b.

FIG. 5a is a perspective view of a medical device according to another embodiment of the invention.

FIG. 5b is a cross-sectional view of the medical device of FIG. 5a taken along line 5b-5b.

FIG. 6a is a perspective view of the medical device according to another embodiment of the invention.

FIG. 6b is a cross-sectional view of the medical device of FIG. 6a taken along line 6b-6b.

### DETAILED DESCRIPTION

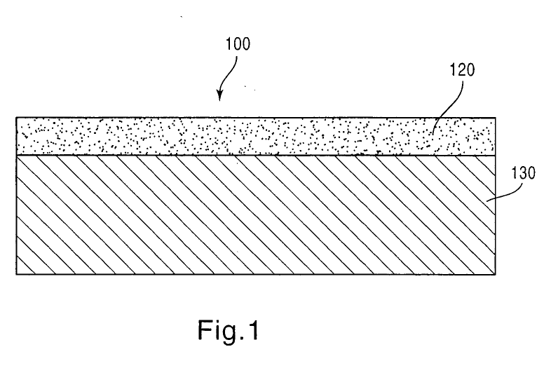

As illustrated schematically in FIG. 1, the medical device 100 includes an agent 120 that is releasably coupled to a carrier 130. The carrier 130 is configured to retain the agent 120 upon the placement of the medical device 100. The agent 120 is configured or formulated to control and/or reduce the inflammation of biological tissue, such as heart tissue. The term “heart tissue” is used herein to mean heart tissue and/or biological tissue surrounding or proximate to the heart, including but not limited to pericardium, epicardium, myocardium, and endocardium.

The carrier 130 is configured to be disposed in operative proximity to biological tissue. In other words, the carrier 130 is configured to be disposed sufficiently close to biological tissue such that the agent 120 may treat the biological tissue. For example, in one embodiment, the carrier 130 is configured to be placed or otherwise disposed proximate to heart tissue.

In one embodiment, the agent 120 is formulated to control and/or reduce inflammation of heart tissue such that the existing myocardial scaffold is not removed or otherwise deteriorated after a myocardial infarct. Accordingly, in such an embodiment, the formation of scar tissue in the heart tissue is controlled and/or reduced.

In one embodiment, the agent 120 is configured to be released from the carrier 130 after the medical device 100 is placed or otherwise disposed proximate to the biological tissue. In another embodiment, the agent 120 is configured to be released from the carrier 130 in a controlled manner. For example, in one embodiment, the agent 120 is configured to be released from the carrier 120 at a constant rate over a period of time. In another embodiment, the agent 120 is configured to be released from the carrier 130 at a first rate for a period of time and at a second rate during another period of time.

In one embodiment, the character of the agent 120 causes the agent 120 to be released from the carrier 130. For example, in such an embodiment, the agent 120 is a coating that is configured to be placed on the carrier and degrade, dissolve, or otherwise separate from the carrier 130 at a constant rate over a period of time. In another embodiment, the carrier 130 is configured to release the agent 120. For example, the agent 120 is disposed in a well of the carrier 130. The carrier 130 includes a well cover that is configured to degrade or dissolve. Thus, when the well cover degrades or dissolves, the agent 120 is delivered to the patient. In another embodiment, the agent 120 is disposed within the carrier 130. The carrier 130 is configured to degrade or dissolve to thereby deliver the agent 120 to the patient.

In one embodiment, the agent 120 includes at least one of the group consisting of NSAIDs, pyrazolones, fenamate, diflunisal, acetic acid derivatives, propionic acid derivatives, oxicam, mefenamic acid, Ponstel, meclofenamate, Meclomen, phenylbutazone, Butazolidin, diflunisal, Dolobid, diclofenac, Voltaren, indomethacin, Indocin, sulindac, Clinoril, etodolac, Lodine, ketorolac, Toradol, nabumetone, Relafen, tolmetin, Tolectin, ibuprofen, Motrin, fenoprofen, Nalfon, flurbiprofin, Ansaid, carprofen, Rimadyl, ketoprofen, Orudis, naproxen, Anaprox, Naprosyn, piroxicam, and Feldene. In another embodiment, the agent 120 includes at least one of the group consisting of mesenchymal stem cells, aspirin in time released form, interleukins, hemeoxygenase, corticosteroids, tacrolimus, and cyclosporine.

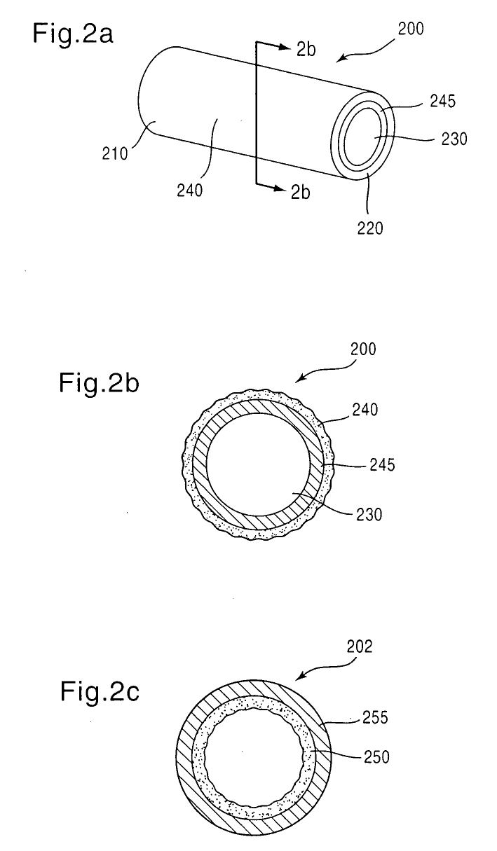

FIG. 2a is a perspective view of a medical device 200 according to an embodiment of the invention. The medical device 200 includes a carrier 245 and an agent 240 releasably coupled to the carrier 245.

In this embodiment, the carrier 245 is a tubular member, such as a stent. The carrier 245 has a first end portion 210 and a second end portion 220. The carrier 245 defines a lumen 230 extending from the first end portion 210 to the second end portion 220. The agent 240 is in the form of a coating that is releasably coupled to an exterior surface of the carrier 245 as shown in FIG. 2b.

The agent 240 may be disposed on or otherwise releasably coupled to the surface of the carrier 245 via any know method, such as a dipping process or a spraying process. See, for example, U.S. Pat. No. 6,569,195, issued on May 27, 2003 and entitled “Stent Coating,” which is hereby incorporated by reference in its entirety.

FIG. 2c is a cross-sectional view of a medical device 202 according to another embodiment of the invention. The medical device 202 includes a tubular carrier 255 and an agent 250. As illustrated, the agent 250 is a coating that is releasably coupled to an inner surface of the carrier 255.

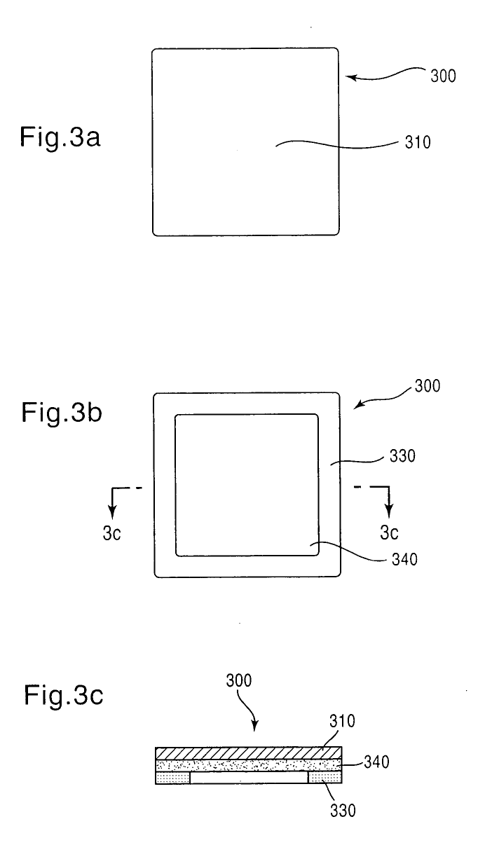

FIG. 3a is a top view of a medical device 300 according to another embodiment of the invention. The medical device 300 includes a carrier 310 and an agent 340. The carrier 310 is configured to be placed on or adhered to surface tissue. The surface tissue may be surface tissue of the patient such as the skin, or surface tissue of the heart.

In the illustrated embodiment, the carrier 310 includes material 330 that is configured to adhere to surface tissue. For example, the material 330 may be an adhesive such as glue. The bottom view of the device 300 is shown in FIG. 3b. As illustrated in FIG. 3b, in this embodiment, the material 330 is disposed along an outer perimeter of the carrier 310. In other embodiments, the material is disposed at other locations of the carrier.

In the illustrated embodiment, the agent 340 is coupled to an underside surface of the carrier 310. Thus, once the agent 340 is released from the carrier 310, the agent 340 contacts and/or penetrates the tissue. In other embodiments, the carrier is configured to release the agent such that the agent may contact and/or penetrate the tissue. A cross-sectional view of the medical device 300 of FIG. 3b taken along line 3c is shown in FIG. 3c. FIG. 3c illustrates the carrier 310 in relation to the agent 340 and in relation to the material 330.

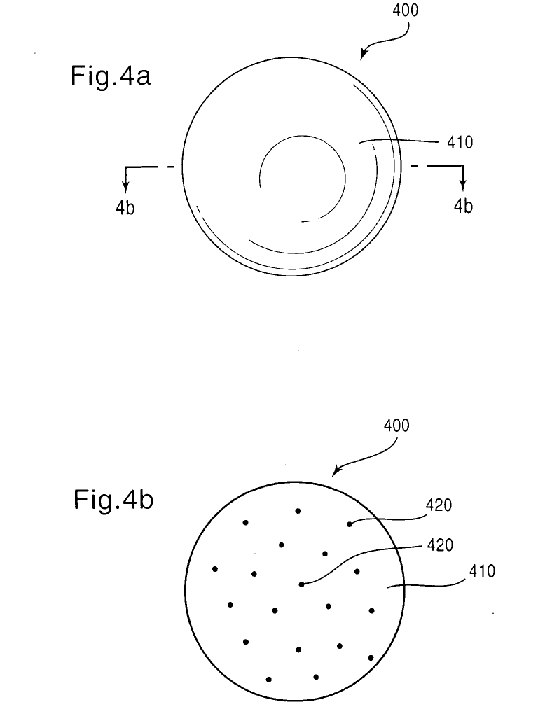

FIG. 4a is a perspective view of a medical device 400 according to another embodiment of the invention. The medical device 400 includes a carrier 410 and an agent 420 releasably coupled to the carrier 410.

In another embodiment, the agent includes the material that is configured to adhere to the surface tissue. In yet another embodiment, the material that is configured to adhere to the surface tissue includes the agent.

In the illustrated embodiment, the carrier 410 is a spherical body or a microsphere. The carrier 410 is configured to degrade in response to the medical device 400 being placed within the body of the patient. The agent 420 is released from the carrier 410 as the carrier 410 degrades.

FIG. 4b is a cross-sectional view of the medical device 400 taken along line 4b-4b in FIG. 4a. The cross-sectional view shows the agent 420 in the carrier 410. Although FIG. 4b shows the agent 420 as granules, it is not necessary that the agent 420 be in granulated form. For example, in alternative embodiments, the agent is a solid, semi-solid, or liquid which fills the inner portion of the microsphere.

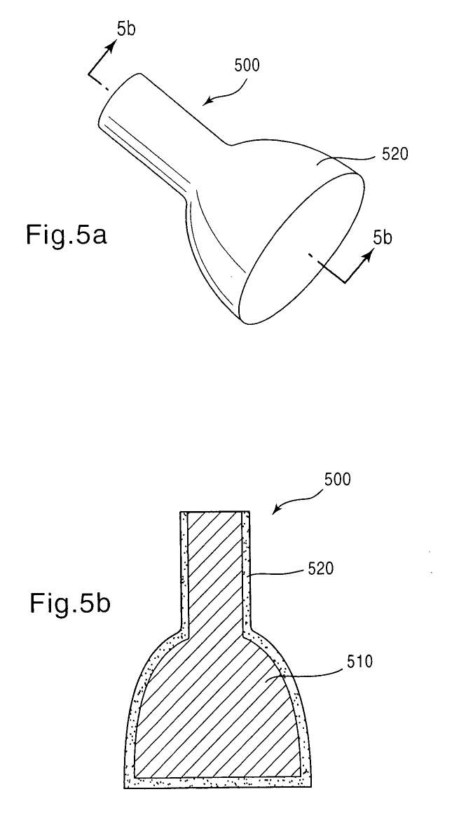

FIG. 5a is a perspective view of a medical device 500 according to another embodiment of the invention. The medical device 500 includes a carrier 510 and an agent 520 releasably coupled to the carrier 510.

In this embodiment, the carrier 510 is configured to be implanted in a body of a patient. For example, in one embodiment, the carrier is an implantable plug. FIG. 5b is a cross-sectional view of the medical device 500 taken along line 5b-5b in FIG. 5a. In this embodiment, the agent 520 is coupled to an exterior surface of the carrier 510.

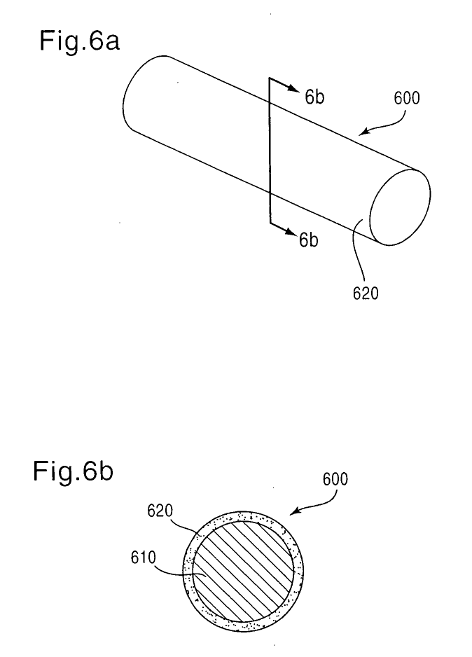

FIG. 6a is a perspective view of a medical device 600 according to another embodiment of the invention. The medical device 600 includes a carrier 610 and an agent 620. FIG. 6b is a cross-sectional view of the medical device 600 taken along line 6b-6b in FIG. 6a. In this embodiment, carrier 610 is a solid tubular structure with the agent 620 coupled to an exterior surface of the carrier 610.

Although the illustrated medical device 500 and 600 illustrate the medical device as having a particular shape, it is not necessary that the medical device be so shaped. In other embodiments, the medical device has a different shape.

In another embodiment of the invention, a medical device has a carrier and an agent releasably coupled to the carrier. In this embodiment, the carrier is a liquid that is configured to solidify in response to being disposed within a body of a patient, such as a solidifying spray solution. The agent is disposed within the carrier. In such an embodiment, the carrier is configured to dissolve or degrade to deliver the agent to the body of the patient. In one embodiment, the carrier is a liquid that is configured to be sprayed or injected into the heart tissue.

In yet another embodiment, a medical device includes an injectable gel or injectable paste that may be injected into a body of a patient.

While the invention has been described in detail and with reference to specific embodiments thereof, it will be apparent to one skilled in the art that various changes and modifications can be made therein without departing from the spirit and scope thereof. Thus, it is intended that the present invention covers the modifications and variations of this invention provided they come within the scope of the appended claims and their equivalents.

What is claimed is:

1. A medical device, comprising:
an agent formulated to control inflammation of heart tissue to prevent the deterioration of myocardial scaffold after a myocardial infarct; and
a carrier to which the agent is releasably coupled, the carrier being configured to be disposed in operative proximity to the heart tissue to be treated by the agent.

2. The device of claim 1, wherein the carrier is a stent.

3. The device of claim 1, wherein the carrier has a first end portion and a second end portion, the carrier defining a lumen extending from the first end portion to the second end portion.

4. The device of claim 1, wherein the carrier is a patch.

5. The device of claim 1, wherein the carrier includes material that is configured to adhere to surface tissue and is configured to release the agent through the surface tissue.

6. The device of claim 1, wherein the carrier includes an adhesive element that is configured to attach the carrier to a body of a patient, the carrier is configured to release the agent through the heart tissue.

7. The device of claim 1, wherein the carrier is a microsphere.

8. The device of claim 1, wherein the carrier includes a spherical body being configured to degrade in response to the medical device being placed within a body of a patient, the agent is configured to be released from the carrier as the spherical body degrades.

9. The device of claim 1, wherein the carrier is a solidifying spray solution.

10. The device of claim 1, wherein the carrier is a liquid that is configured to solidify in response to being disposed within a body of a patient.

11. The device of claim 1, wherein the carrier is an injectable gel.

12. The device of claim 1, wherein the carrier is an injectable paste.

13. The device of claim 1, wherein the carrier is a semi-solid material that is configured to be injected into a body of a patient.

14. The device of claim 1, wherein the carrier is an implantable plug.

15. The device of claim 1, wherein the carrier is a body of material that is configured to be implanted in a body of a patient.

16. The device of claim 1, wherein the carrier is configured to release the agent in a controlled manner.

17. The device of claim 1, wherein the agent is configured to be released from the carrier in a controlled manner.

18. The device of claim 1, wherein the agent is configured to be released from the carrier at a first rate for a first period of time and at a second rate, different than the first rate, for a second period of time, different than the first period of time.

19. The device of claim 1, wherein the agent includes at least one of the group consisting of: mesenchymal stem cells, aspirin in time released form, interleukins, hemeoxygenase, corticosteroids, tacrolimus, and cyclosporine.

20. The device of claim 1, wherein the agent includes at least one of the group consisting of: NSAIDs, pyrazolones, fenamate, diflunisal, acetic acid derivatives, propionic acid derivatives, oxicam, mefenamic acid, Ponstel, meclofenamate, Meclomen, phenylbutazone, Butazolidin, diflunisal, Dolobid, diclofenac, Voltaren, indomethacin, Indocin, sulindac, Clinoril, etodolac, Lodine, ketorolac, Toradol, nabumetone, Relafen, tolmetin, Tolectin, ibuprofen, Motrin, fenoprofen, Nalfon, flurbiprofin, Ansaid, carprofen, Rimadyl, ketoprofen, Orudis, naproxen, Anaprox, Naprosyn, piroxicam, and Feldene.

21. A medical device, comprising:
an agent formulated to control inflammation of biological tissue to prevent the formation of scar tissue; and
a carrier to which the agent is releasably coupled, the carrier being configured to be disposed in operative proximity to the biological tissue to be treated by the agent.

22. The device of claim 21, wherein the carrier is a stent.

23. The device of claim 21, wherein the carrier is a patch.

24. The device of claim 21, wherein the carrier is a microsphere.

25. The device of claim 21, wherein the carrier is a solidifying spray solution.

26. The device of claim 21, wherein the carrier is an injectable gel.

27. The device of claim 21, wherein the carrier is an injectable paste.

28. The device of claim 21, wherein the carrier is an implantable plug.
